(19) United States
(12) Patent Application Publication
(10) Pub. No.: US 2026/0266780 A1
(43) Pub. Date: Sep. 10, 2026

(54) MEDIUM IDENTIFICATION METHOD AND DEVICE

(71) Applicant: The Hong Kong Polytechnic University, Kowloon (HK)

(72) Inventors: Juncen Zhu, Kowloon (HK); Jiannong Cao, Kowloon (HK)

(21) Appl. No.: 19/073,292

(22) Filed: Mar. 7, 2025

(51) Int. Cl. G01N 30/86; G01N 29/46; G01N 30/76; G01N 33/14

(52) U.S. Cl. CPC G01N 30/8662; G01N 29/46; G01N 30/76; G01N 33/146

(57) ABSTRACT

This application relates to a method and system for identifying a medium, comprising: receiving one or more signals emitted from one or more signal sources and transmitted through the medium; extracting the frequency-dependent attenuation characteristics of the medium from the signals; obtaining mass spectra from the frequency-dependent attenuation characteristics of the medium by utilizing a Conditional Generative Adversarial Network; identifying the medium using a trained convolutional neural network based on the mass spectra. The system and method of this application can identify counterfeit and adulterated substances without opening containers, demonstrating exceptional levels of accuracy, precision, and resilience across various liquid categories, storage environments, and container compositions.

### TECHNICAL FIELD

The present invention relates to signal processing technology, and more particularly to an acoustic and mass spectrometry-based medium identification method and device.

### BACKGROUND

Counterfeit and adulterated liquids pose significant threats to public health and economic stability, resulting in numerous poisoning incidents and substantial economic losses annually. Detecting and verifying liquid authenticity is crucial; however, traditional methods such as chemical and chromatographic techniques, Quasi-Static Electrical Tomography (QET), and surface tension measurement, require opening containers and specialized equipment. These methods not only compromise product integrity but also increase operational costs making them less practical for large-scale or real-time applications. While effective for detecting contaminants, these intrusive methods often fail to provide scalable, non-destructive solutions. Recent advancements in non-invasive technologies, including RFID-based systems, ultra-wideband (UWB) approaches, and millimetre wave (mm Wave) radar systems like FG-LiquID, offer promising alternatives by preserving container integrity. However, these systems can be highly susceptible to environmental interference, require specialized and costly hardware, and struggle with fine-grained liquid authentication, limiting their practical scalability.

Acoustic-based methods provide a cost-effective, non-invasive alternative by leveraging the unique absorption and transmission characteristics of sound waves in liquids. While promising, current acoustic methods face significant challenges related to variations in container materials, shapes, and environmental conditions. Moreover, these methods lack the molecular-level precision required for definitive liquid identification, which affects both accuracy and reliability. Additionally, although the absorption rate of sound waves provides some insight into liquid characteristics, it fails to offer the interpretability and specificity of chemical analysis techniques, reducing its potential for high-confidence identification. This limitation may reduce reliability of the detection technology.

Therefore, there is a compelling need for a liquid authentication method and system that ensures high accuracy, precision, and robustness across diverse liquid types, storage conditions, and container materials, making it suitable for real-world, large-scale, and industrial applications.

### SUMMARY

According to the first aspect of the present application, there is provided a method for identifying a medium, comprising: receiving one or more wave-based signals emitted from one or more signal sources and transmitted through the medium; extracting wave propagation characteristics of the medium from the signals; transforming the extracted characteristics into a multi-dimensional spectral representation through computational analysis; identifying the medium with a trained analysis system at least based on the generated spectral representation.

In a preferred embodiment, the step of receiving one or more wave-based signals further comprising capturing the signals with an array of spatially distributed signal receivers, selecting the captured signals for weighted fusion and then performing the weighted fusion of selected captured signals to obtain at least one fused signal.

Preferably, the weight assigned to each selected signal for signal fusion is determined with a normalized signal-to-noise ratio approach. Advantageously, the weight assigned to each selected signal is computed as the ratio of its signal-to-noise ratio to the total signal-to-noise ratio of all selected captured signals. Advantageously, only some of the received signals or the most relevant to the influenced most by the medium.

Preferably, the array of spatially distributed signal receivers is configured as a cross-shaped array, circular array, or spherical array.

In a further preferred or an alternative embodiment, the method further comprises conducting signal processing including noise reduction and/or de-reverberation on the signals before extracting the wave propagation characteristics of the medium from the signals.

Preferably, the noise reduction is conducted through a denoising algorithm employing a discrete wavelet transform, and the de-reverberation is conducted through a weighted prediction error algorithm.

In a further preferred or an alternative embodiment, the one or more wave-based signals are acoustic signals, and the extracting step further includes performing a fast Fourier transform on the fused signal, extracting a frequency domain amplitude at each frequency, and obtaining an acoustic absorption and transmission curve as the wave propagation characteristics of the medium by normalising the extracted frequency domain amplitude by amplitudes of originally emitted wave-based signals.

In a further preferred or an alternative embodiment, the method further comprises using a container compensation model to correct and compensate for the wave propagation characteristics of the medium accommodated in a container.

Preferably, the container compensation model utilizes a container transfer function defined as the ratio of a wave propagation characteristics of the medium with container influence to a wave propagation characteristic of the medium without container influence to perform the compensation. Preferably, the container transfer function is learned through training a neural network regression model designed to transfer container characteristics, and wherein the neural network regression model includes an input layer receiving container characteristics, hidden layers incorporating nonlinear activation functions, and an output layer generating estimated wave propagation characteristics, with mean squared error as the loss function for training.

In a further preferred or an alternative embodiment, the computational analysis is conducted through a deep learning model including conditional generative adversarial network, or autoencoders or Transformer-based networks.

Preferably, the conditional generative adversarial network comprises a generator and discriminator, wherein the generator is configured to use the wave propagation characteristics as input to produce mass spectra as the spectral representation, and the discriminator is configured to classifies mass spectra based on the wave propagation characteristics of the medium. Advantageously, the conditional generative adversarial network effectively translates extracted characteristics features into mass spectra, enabling molecular-level identification of the medium.

In a further preferred or an alternative embodiment, the trained analysis system includes a fully connected neural network comprising an architecture with an input layer, three hidden layers, and an output layer, and wherein the input layer receives a spectrum vector representing the intensity of a specific mass-to-charge ratio, the three hidden layers consist of 512, 256, and 128 neurons, each using the ReLU activation function to introduce nonlinearity, and the output layer uses a Softmax activation function to output a probability distribution over the identification.

In any of the aforementioned embodiments, the medium may consist of a liquid, solid, or a mixture of liquid and solid. The wave-based signals may be acoustic signals or any other mechanical wave-based signals, and the wave propagation characteristics of the medium may include frequency-dependent attenuation characteristics like acoustic absorption and transmission curves. The multi-dimensional spectral representation may be mass spectra.

According to the second aspect of the present application, there is provided a device or system for identifying a medium, comprising a processor configured to execute any one of the methods as described above in the first aspect of the present application.

Preferably, the device or system for identifying a medium further comprises an array of signal receivers for capturing signals transmitted through the medium.

According to the third aspect of the present application, there is provided a system for identifying a medium, comprising: a receiver module/unit for receiving one or more wave-based signals emitted from one or more signal sources and transmitted through the medium; an extraction module/unit for extracting wave propagation characteristics of the medium from the signals; a transformation module/unit for transforming the extracted characteristics into a multi-dimensional spectral representation through computational analysis; an identification module/unit for identifying the medium with a trained analysis system at least based on the generated spectral representation.

According to the fourth aspect of the present application, there is provided a non-transitory computer readable medium storing a plurality of instructions that when executed control a computer including one or more processors to implement the methods as described above in the first aspect of the present application.

The system and method of this application are designed to identify counterfeit and adulterated liquids without requiring container opening. By analysing AATC through liquids and correlating them with mass spectrometry data using a cGAN, this application extracts molecular-level characteristics to enable precise identification. This system employs a cross-shaped microphone array to minimize signal variability, an adaptive container compensation model that adjusts for different container shapes and materials, and advanced neural network(s) for generating mass spectra and liquid classification. Experimental assessments have demonstrated exceptional levels of accuracy, precision, and resilience across various liquid categories, storage environments, and container compositions, establishing the system as a reliable solution for real-world liquid authentication needs across a wide range of industries.

In particular, while many non-invasive liquid authentication systems rely solely on acoustic signatures, lacking molecular-level precision, the incorporation of cGAN in this application bridges this gap, offering an advantageous method and system for achieving high accuracy and detailed identification. Through the mapping of these signals to mass spectra, the system achieves high-resolution identification capabilities comparable to chemical analysis, all without the need to physically open the container.

Moreover, the utilization of a cross-shaped microphone array design ensures that signals are captured with minimal distortion from various angles, effectively addressing common issues such as multipath interference. This design enhancement enhances system robustness and accuracy across diverse setups and conditions.

Furthermore, the system offers enhanced container flexibility. Additionally, compared to current solutions that suffer from constraints due to sensitivity to container materials or shapes, the container compensation model in this system allows for adaptable use across various packaging materials and configurations without compromising accuracy.

In addition, the system ensures improved signal reliability in noisy environments. Comparing with existing methods that often struggle in noisy or reverberant conditions, leading to reduced reliability, the integrated signal processing in this application provides consistent performance even in less controlled environments, making it a more dependable choice for industrial or field applications. Through the implementation of noise reduction and dereverberation techniques, the system guarantees that signals remain clear and interpretable even in complex environments characterized by echo or background noise. This capability is vital for real-world applications where environmental control is limited, ensuring reliable performance in varied operational settings.

Therefore, the present application provides a more accurate solution compared to traditional non-invasive techniques.

It should be understood that medium identified by the method and system according to preferred embodiments of the present application includes a liquid or combination of various liquid having different characteristics, e.g., wine, beer, Chinese white spirit, brandy, whisky, vodka. Furthermore, the method and system according to preferred embodiments of the present application could be further applied to identify other medium including fluid and solid materials (e.g., powders, grains) by adapting the acoustic processing to detect other physical characteristics.

A person skilled in the art would understand that the described embodiments may be implemented through hardware, software or a combination of both. This program can be stored in a computer-readable storage medium, such as read-only memory, a magnetic disk, or an optical disk. The described embodiments are optional and not meant to limit this application. Any modifications, equivalent replacements, improvements, etc., made within the spirit and principle of this application are included within its scope of protection.

### BRIEF DESCRIPTION OF THE DRAWINGS

The accompanying figures, which are incorporated in and constitute a part of this specification, illustrate one or more embodiments of the invention and, together with the description, serve to explain the principles of the invention.

FIG. 1 shows a system for identifying a medium according to an embodiment of the present application.

FIGS. 2A-2C show an exemplary configuration of a cross-shaped array comprises five microphones according to an embodiment of the present application.

FIGS. 3A and 3B show an illustrative hardware configuration according to an embodiment of the present application.

FIGS. 4A-4B show an illustrative test device according to an embodiment of the present application.

FIG. 5 shows the process of identifying the medium implemented by the embodiments of FIGS. 1, 3A and 3B and 4A-4B.

FIG. 6 shows the accuracy of brand recognition under normal and abnormal storage conditions for diverse beverage varieties according to an embodiment of the present application.

FIG. 7 illustrates accuracy of production year verification for wine across diverse vintages and storage conditions according to an embodiment of the present application.

FIG. 8 illustrates accuracy of production year verification for Chinese white spirit across various vintages and storage conditions according to an embodiment of the present application.

FIG. 9 shows accuracy without container compensation for adulteration under normal and improper storage conditions.

FIG. 10A shows accuracy with single microphone and speaker under normal and improper storage conditions.

FIG. 10B shows accuracy with microphone array under normal and improper storage conditions.

### DETAILED DESCRIPTION

Detailed reference is now made to the embodiments of the present application, with the figures illustrating one or more embodiments. The repeated use of figure labels throughout this specification serves to indicate similar features or elements of the present application. The following content is provided to facilitate a further understanding of the present application for those skilled in the art but does not limit the application in any form. It should be noted that various modifications and changes can be made by those skilled in the art without departing from the concept of the present application. For example, features shown or described as part of one embodiment may be used in conjunction with another embodiment to generate further implementations. Consequently, the present application is intended to encompass such variations and changes within the scope of the appended claims and their equivalents.

FIG. 1 illustrates a medium identification system 110 for identifying a medium, e.g., a liquid. This system is applicable in various scenarios, including detecting industrial alcohol adulteration, identifying counterfeit brands, verifying production years, and authenticating liquid products. In an exemplary embodiment of the present application, the system is used as a liquid authentication system.

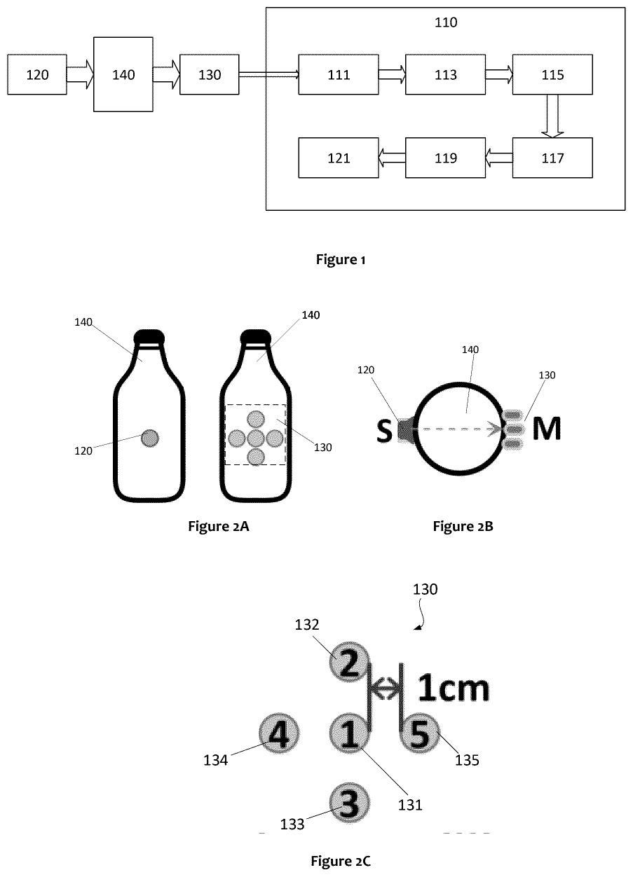

As shown in FIG. 1, the medium identification system 110 receives signals from the receiver array 130, which could be an array of microphones 131 arranged in a cross shape, as shown in FIG. 2A-2C, to receive signals originally emitted from the signal source 120, which could be a speaker. The acoustic signal emitted from the speaker 120 is transmitted through the liquid to be classified and identified accommodated in the container 140 and arrives at the receiver array 130.

In a specific embodiment illustrated in FIGS. 2A-2C, a configuration of a cross-shaped array 130 comprises five microphones 131-135 positioned around the container: Microphone 131 is oriented directly towards the speaker 120, aligned with the container's maximal diameter; Microphones 132 and 133 are situated directly above and below 131, respectively; Microphones 134 and 135 are positioned to the left and right of 131, correspondingly. The spacing between each microphone is preferably 1 cm. This configuration of the cross-shaped microphone array 130 can alleviate signal distortion and attenuation caused when sound waves pass through the container.

In particular, when sound waves travel within a liquid container 140, interactions with the container walls and the liquid lead to reflections and scattering phenomena. These multipath phenomena introduce multiple instances of the transmitted signal, arriving at different times and amplitudes at the microphones. Such distortions complicate the accurate extraction of the frequency-dependent attenuation characteristics, including AATC.

The cross-shaped microphone array 130 is specifically designed to counteract these effects by ensuring uniform path lengths from the speaker 120 to each microphone, thereby reducing time delays and amplitude variations. Microphone 131 captures the direct signal, while microphones 132 to 135 capture signals influenced by multipath effects.

The signals initially captured by the microphone array 130 are then fed into the medium identification system 110 for further processing and identification.

In a preferred embodiment, the medium identification system 110 comprises a signal collection and fusion module/unit (111) responsible for consolidating signals captured by the microphone array 130 into a cohesive fused signal for subsequent analysis. Once fused, the signals are processed by the signal processing module 113, which applies noise reduction and dereverberation techniques to refine data quality. The system further integrates an extraction module/unit (115) designed to extract the acoustic attenuation time curve (AATC) from the processed signal, enabling detailed analysis of attenuation characteristics. Additionally, a container compensation module/unit (117) is included to address attenuation effects induced by the container, thereby refining the AATC data. Following compensation, the mapping module 119 associates the corrected AATC data with corresponding mass spectra, utilizing advanced modeling techniques to reveal molecular-level composition. Finally, an identification module/unit (121) is incorporated to apply advanced algorithms for the accurate identification and classification of the medium based on the processed data and mass spectra. A comprehensive discussion of these modules is provided in subsequent sections, noting that variations in implementation may allow selective incorporation of specific modules based on operational needs.

### Signal Collection and Fusion Module/Unit 111

Referring back to FIG. 1, a signal collection and fusion module/unit 111 of system 110 is designed for collecting and fusing signals from microphones 131-135. Utilizing signals from multiple microphones exploits spatial diversity to enhance signal quality. Weighted fusion of these signals is conducted, with weights [symbol] ᵢ assigned based on the signal-to-noise ratio (SNR) of each microphone. This process amplifies the desired signal s_{liquid}(t) while suppressing noise nᵢ(t) and undesired reflections s_{container}(t).

The signal received at microphone (i) is represented by Equation 1,

$${x}_{i} ( t ) = {s}_{liquid} ( t - {τ}_{i} ) + {s}_{container} ( t - {τ}_{i} ) + {n}_{i} ( t ) \qquad ( 1 )$$

where τᵢ accounts for time delays arising from positional differences.

The fused signal is computed, as per Equation 2,

$${𝒳}_{fused} ( t ) = {\sum }_{i = 1}^{5} {𝒲}_{i} {𝒳}_{i} ( t ) \qquad ( 2 )$$

with weights determined by Equation 3:

$${𝓌}_{4} = \frac{{SNR}_{i}}{{\sum }_{j = 1}^{5} {SNR}_{i}} \qquad ( 3 )$$

This approach mitigates multipath interference and positional discrepancies, thereby enhancing the resilience and precision of signal acquisition AATC extraction.

### Signal Processing Module/Unit 113

To address the limitations of existing methods that struggle in noisy or reverberant conditions and noisy environments, resulting in poor reliability, the media identification system 110 incorporates noise reduction and dereverberation techniques to enhance reliability even in challenging environments with echoes or background noise. These techniques improve reliability by ensuring that signals remain clear and interpretable, particularly in scenarios where environmental control is limited. The use of integrated signal processing techniques guarantees consistent performance, making the system a reliable choice for industrial or field applications.

In a preferred embodiment, the signal processing module/unit (113) is specifically designed for noise reduction and dereverberation purposes. The inclusion of this module is optional and can be adjusted or omitted depending on the environmental conditions, particularly in low-noise settings where minimal interference is present.

In the preferred embodiment, the fused signal is directed to the signal processing module/unit 113 for noise reduction and dereverberation to ensure the fidelity of the resulting fused signal χ_{fused}(t) in representing the liquid's acoustic characteristics.

To effectively suppress environmental noise and electromagnetic interference, a denoising algorithm based on the Discrete Wavelet Transform (DWT) is applied. By decomposing the signal into multiple frequency bands and applying thresholding to wavelet coefficients, this method reduces noise while preserving critical signal features, making it particularly effective in non-stationary noise environments. This method is suitable for non-stationary noise environments.

Additionally, reverberation artifacts caused by reflections within the container are mitigated using the Weighted Prediction Error (WPE) algorithm. By modeling and subtracting late reverberation components, the system enhances the direct-path signal, improving temporal resolution and spectral clarity, which are crucial for accurate frequency domain analysis and for minimizing spectral feature smearing.

In an alternative embodiment, the sequence of signal processing and fusion steps can be interchanged, that is, the initial processing of signals received from the microphone array 130 to reduce noise is conducted before carrying out the fusion step.

### Extraction Module/Unit 115

The extraction module/unit 115 is used to extract acoustic features that can be used for liquid identification.

When acoustic waves propagate through a liquid, they interact with the molecular structure of the liquid, thereby influencing the absorption and transmission characteristics of the waves. The acoustic impedance of a liquid is a function of its density and sound propagation velocity, both of which are intrinsically linked to the liquid's molecular composition. Variations in molecular structure result in differences in viscosity and elasticity, subsequently impacting the acoustic absorption coefficient at a frequency. Through the analysis of the frequency-dependent acoustic signal, the molecular properties of the liquid can be obtained.

One of the key features utilized for liquid identification and authentication is the AATC, which is derived from the acoustic signal's interaction with the liquid during propagation. The AATC effectively captures essential acoustic characteristics crucial for liquid authentication. This curve reflects the frequency-dependent absorption properties of the liquid, a quality directly influenced by its unique molecular composition.

In the preferred embodiment, AATC is extracted and utilised. In particular, after the processing including noise reduction and dereverberation, the AATC, a feature generated from the acoustic signal as it travels through the liquid, is extracted to capture the liquid's frequency-dependent attenuation characteristics in the extraction module/unit 115 through the following steps:
1) Frequency Domain Conversion: A Fast Fourier Transform (FFT) is performed on χ_{fused}(t) to obtain χ_{fused}(ƒ).
2) Amplitude Extraction: The amplitude spectrum R(ƒᵢ)=|X_{fused}(ƒᵢ)| is extracted at frequencies ƒᵢ from 18 kHz to 20 KHz.
3) Normalization: R(ƒᵢ) is normalized by the known amplitudes of the emitted signal S(ƒᵢ) to obtain:

$$AATC ( {f}_{i} ) = \frac{R ( {f}_{i} )}{S ( {f}_{i} )} \qquad ( 4 )$$

where S(ƒᵢ)=Aᵢ, accounting for the amplitudes of the emitted signal at frequencies ƒᵢ.

The emitted acoustic signal s(t) consists of multiple sine waves with amplitudes Aᵢ at different frequencies ƒᵢ ranging from 18 kHz to 20 kHz:

$$s ( t ) = {\sum }_{i = 1}^{n} {A}_{i} sin ( 2 π {f}_{i} t ) \qquad ( 5 )$$

where [symbol] is the number of discrete frequencies. These high frequencies are chosen to minimize background noise and human voice interference.

The AATC effectively reflects how various substances absorb acoustic energy at different frequencies, which is influenced by molecular composition, molecular size and bonding properties. This analysis serves as the foundation for subsequent molecular composition identification.

### Container Compensation Module/Unit 117

The preferred embodiment further includes a container compensation module/unit 117 to address the variability introduced by different container materials and shapes.

In particular, upon obtaining the AATC that characterizes the frequency-dependent attenuation traits of the liquid, the container's material properties (e.g., density, elasticity), shape, and thickness, can significantly influence the transmission and attenuation of acoustic signals. These effects have the potential to obscure or distort the genuine acoustic fingerprint of the liquid, resulting in inaccurate AATC readings.

The container compensation module/unit 117 is deployed to rectify the acquired AATC.

The container compensation model embedded in the container compensation module/unit 117 quantifies the container's impact and adjusts the observed AATC accordingly. A comprehensive dataset of AATC data is collected from experiments involving diverse containers constructed from various materials (such as glass, plastic, metal) and with different shapes (including cylindrical, rectangular, irregular), capturing the range of container effects on acoustic signal transmission.

By collecting data from various containers and applying machine learning algorithms, the container compensation module/unit 117 adjusts the AATC to minimize the container's impact on the acoustic measurements.

The container transfer function H_{c}(ƒ) is defined as the ratio of the observed AATC with the container to the ideal AATC devoid of container influence, represented by Equation 4.

$${H}_{c} ( {AATC}_{container + liquid} ( f ) ) = {AATC}_{liquid} ( f ) \qquad ( 6 )$$

Here, AATC_{container+liquid}(ƒ) denotes the measured AATC when the liquid is enclosed in the container, while AATC_{liquid}(ƒ) signifies the intrinsic AATC of the liquid derived from reference measurements (e.g., in a standardized container or simulation).

A regression model, specifically a neural network regression model, is trained to learn H_{c}(⋅) for transferring container features (material properties, dimensions). The neural network is structured with input nodes for the container characteristics, hidden layers featuring nonlinear activation functions, and an output layer generating the estimated AATC_{liquid}(ƒ). Mean squared error (MSE) is utilized as the loss function during training.

This compensation process effectively eliminates the container's influence, enabling the corrected AATC_{liquid}(ƒ) to more precisely mirror the authentic acoustic properties of the liquid.

It should be understood that an alternative embodiment may not include container compensation module/unit 117.

### Mapping Module/Unit 119

Following the compensation of the AATC, the compensated AATC is mapped to the corresponding mass spectra within the mapping module/unit 119 utilizing a deep learning model. The deep learning model could be the cGANs. Alternatively, the deep learning model could also be embodied as autoencoders or Transformer-based networks.

The adjusted AATC characteristics are input into a pre-trained cGAN, which facilitates the mapping to associated mass spectra. This mapping mechanism enables the extraction of molecular-level insights from acoustic data, thereby enhancing the interpretive depth and robustness of the system. The purpose and principle are explained below.

Matrix-Assisted Laser Desorption/Ionization Time-of-Flight (MALDI-TOF) mass spectrometry offers detailed information about the molecular constitution of a sample through the measurement of the mass-to-charge ratio (m/z) of ionized particles. Each liquid possesses a unique mass spectrum that functions as a unique molecular identifier.

The correlation between acoustic absorption properties and mass spectra resides in their sensitivity to the molecular composition of a liquid. Both parameters react to variations in molecular structures, impacting characteristics such as density, viscosity, and elasticity, thereby affecting acoustic absorption and ionization patterns in mass spectrometry. While AATC conveys macroscopic acoustic insights influenced by molecular attributes, mass spectrometry provides microscopic details on molecular composition. This correlation is viable due to the alignment between alterations in acoustic characteristics, impacted by molecular composition, and the molecular patterns identified in mass spectrometry. This process enables the deduction of molecular-level details from acoustic data, thereby amplifying the interpretative capacity and precision of liquid authentication.

The mapping module/unit 119 utilises a data-driven approach employing cGANs to correlate the AATC with the associated mass spectra.

Directly linking acoustic attenuation traits with mass spectra is intricate due to the nonlinear and multi-dimensional interrelations involved. Hence, the preferred embodiment involves employing a data-centric approach utilizing cGANs to establish a mapping between the AATC and the corresponding mass spectra.

The cGAN, an extension of the conventional GAN, conditions the generation process on additional information (in the preferred embodiment, the AATC). It comprises two primary components:
Generator G: A neural network that takes the AATC x as input and generates a simulated mass spectrum ŷ=G(x).
Discriminator D: A neural network that assesses the authenticity of the mass spectrum given the AATC, delivering a probability that a pair (x, y) is genuine or falsified.

A dataset of paired AATC and corresponding mass spectra obtained from controlled experiments is curated. The mass spectra undergo preprocessing to align peaks and normalize intensities, ensuring uniformity across samples.

The generator and discriminator undergo simultaneous adversarial training. The generator G strives to produce mass spectra that are indiscernible from genuine ones, given the input AATC. Concurrently, the discriminator D endeavors to accurately differentiate between genuine and generated mass spectra, conditioned on the AATC. Consequently, the generator becomes proficient in inferring mass spectra from the AATC accurately.

The loss functions used are:
Adversarial loss for D:

$${ℒ}_{D} = - {𝔼}_{x , y} [ log D ( x , y ) ] - {𝔼}_{x} [ log ( 1 - D ( x , G ( x ) ) ) ] \qquad ( 6 )$$

Adversarial loss for G:

$${ℒ}_{GAN} = - {𝔼}_{x , y} [ log D ( x , G ( x ) ) ] \qquad ( 7 )$$

Reconstruction loss:

$${ℒ}_{MSE} = {𝔼}_{x , y} [ {‖ y - G ( x ) ‖}^{2} ] \qquad ( 8 )$$

Total loss for G:

$${ℒ}_{G} = {ℒ}_{GAN} + {λℒ}_{MSE} \qquad ( 9 )$$

where λ is a hyperparameter that balances the adversarial and reconstruction losses.

The optimization of G and D is executed through the Adam optimizer with a learning rate of 1×10⁻⁴, alternatingly enhancing G and D. The networks undergo training for 200 epochs with a batch size of 64, minimizing the respective loss functions [symbol] _{G} and [symbol] _{D}.

The cGAN framework facilitates capturing the intricate, nonlinear mapping from AATC to mass spectra without explicitly modelling the underlying physics.

### Identification Module/Unit 121

Upon obtaining the mass spectra from the AATC, the identification module/unit 121 processes the mass spectra using a trained convolutional neural network to process the mass spectra to identify the liquid, e.g., to ascertain the authenticity and categorization of the liquid based on its molecular composition and spectral characteristics.

In an exemplary embodiment, the identification of the liquid includes fraud detection, brand detection and year verification. In particular, exemplary embodiments of the present application can be used to detect fraudulent practices related to industrial alcohol across various beverage types. They can also distinguish between different beverage brands through their distinct molecular compositions, even under varying storage conditions. Furthermore, they can verify production years for products like wine and Chinese white spirit, spanning different vintages and storage environments.

The mass spectra generated encapsulated comprehensive details regarding the molecular structure of the liquid. The identification module employs a Fully Connected Neural Network (FCNN) to analyze these spectra and classify liquids into categories such as authentic versus counterfeit, specific brands, or production years.

The architecture of the FCNN comprised:
Input Layer: Receiving the mass spectrum vector where each element denoted the intensity at a specific mass-to-charge ratio (m/z value).
Hidden Layers: A network design featuring three hidden layers with 512, 256, and 128 neurons respectively. Each layer incorporated the ReLU activation function to introduce nonlinearity.
Output Layer: Generating a probability distribution across classes through a Softmax activation function for multi-class classification.

The network underwent training utilizing the cross-entropy loss function, quantifying the deviation between predicted probabilities and actual labels. Parameter updates are executed using the Adam optimizer with a learning rate set at 1×10⁻⁴ to minimize loss across the training dataset. Techniques like batch normalization are applied post each hidden layer to stabilize and hasten training.

Performance evaluation of the FCNN encompassed metrics such as accuracy, precision, recall, and F1-score on a validation dataset. Enhanced performance indicated the FCNN's proficiency in differentiating between various liquid classes based on the mass spectra.

Functioning as the ultimate decision-making element within the framework, the FCNN leveraged the intricate molecular insights derived from the mass spectra to deliver precise authentication and classification of liquids, effectively tackling the complexities introduced by adulteration and counterfeiting practices.

It should be noted that the aforementioned depiction illustrates a preferred embodiment of the current invention, and alternative versions may involve only a subset of these modules or components for identifying a target liquid or other medium in a less noisy environment or in straightforward cases.

For example, in certain situations focused on liquid identification and authentication, it may be possible to incorporate solely four modules or units: the signal collection module/unit 111 for receiving signals from the microphone or receiver; the extraction module/unit 115 for extracting the frequency-dependent attenuation characteristics of the medium from the received signals; the mapping module/unit 119 for deriving mass spectra from the frequency-dependent attenuation characteristics of the medium through the utilization of a deep learning model; and the identification module/unit 121 for conducting the identification of the medium by utilizing a trained convolutional neural network based on the obtained mass spectra.

Moreover, it is feasible to selectively include one or more of the signal processing module/units 113 and the container compensation module/unit 117 according to specific industrial needs to optimize overall system performance.

It should be noted that the embodiments described above are illustrative; units or modules described as separate may or may not be physically distinct, and components displayed as units or modules may or may not be physical units. They may be located in one place or distributed across multiple network units.

In the device embodiments provided in this application, the connection relationships between modules in the accompanying drawings indicate communication links, which could be implemented as one or more communication buses or signal lines.

Based on the description of the implementations above, those skilled in the art would understand that all or part of the units or modules for implementing the described embodiments can be completed by hardware or by a program instructing related hardware. This program can be stored in a computer-readable storage medium, such as read-only memory, a magnetic disk, or an optical disk. The described embodiments are optional and not meant to limit this application. Any modifications, equivalent replacements, improvements, etc., made within the spirit and principle of this application are included within its scope of protection.

In most cases, the essence of the technical solution or the part contributing to the existing technology in this application can be embodied in the form of a software product. This computer software product is stored in readable storage media such as computer floppy disks, USB drives, external hard drives, read-only memory (ROM), random access memory (RAM), disks, or CDs, containing instructions to enable a computer device (which could be a personal computer, training device, or network device) to execute the methods described in the various embodiments of this application.

In the aforementioned embodiments, software, hardware, firmware, or any combination thereof can be used to fully or partially achieve the implementation. When software is used, it can be implemented wholly or partially in the form of a computer program product.

The computer program product comprises one or more computer instructions. When these computer program instructions are loaded and executed on a computer, they fully or partially generate the processes or functions as described in the embodiments of this application. The computer can be a general-purpose computer, a special-purpose computer, a computer network, or any other programmable device. The computer instructions can be stored in computer-readable storage media or transferred from one computer-readable storage medium to another, such as from a website, computer, training device, or data center to another site, computer, training device, or data center via wired (e.g., coaxial cables, fiber optics, digital subscriber lines) or wireless (e.g., infrared, wireless, microwave) means. The computer-readable storage medium can be any available medium that a computer can store data on, or a data storage device that integrates one or more available media, such as magnetic media, optical media, or semiconductor media.

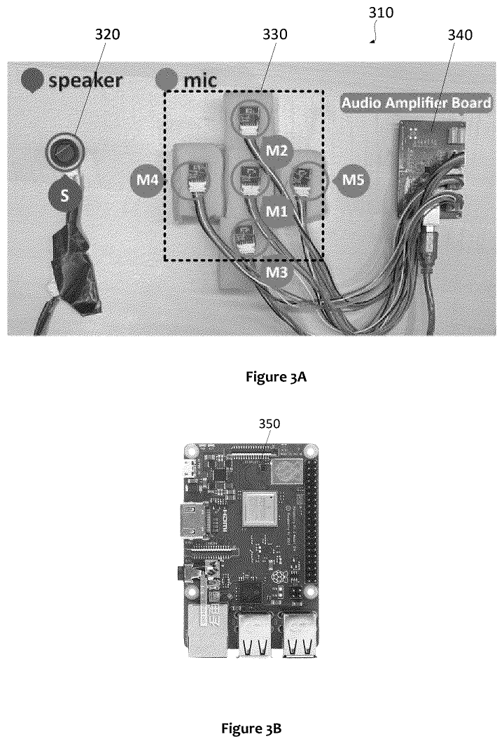

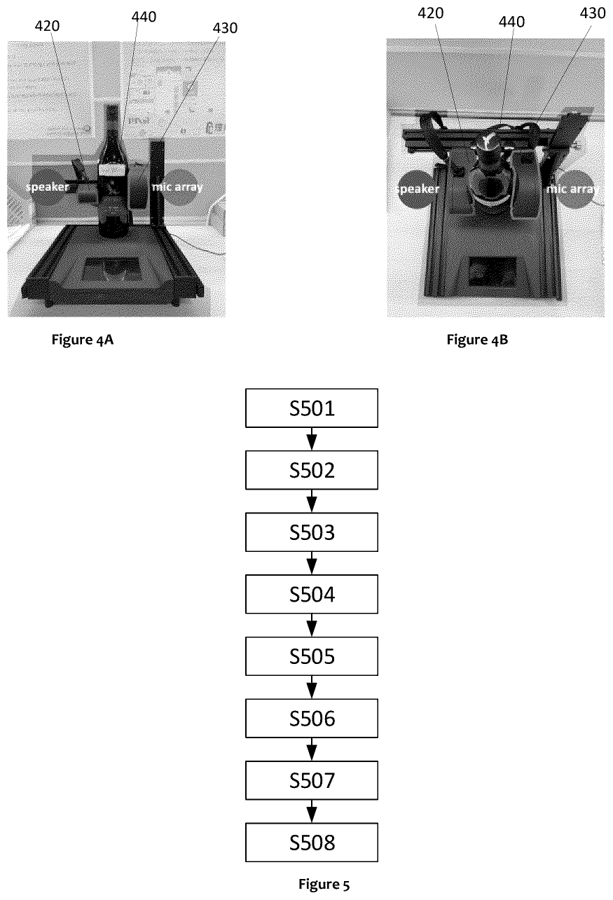

FIGS. 3A and 3B show an illustrative hardware configuration 310 according to anther embodiment of the present application. FIGS. 4A-4B show an illustrative test device 410 according to anther embodiment of the present application.

The hardware configuration 310, illustrated in FIGS. 3A and 3B, includes a speaker 320, a microphone array 330, an amplifier board 340, a Raspberry Pi 3B 350, and an UltrafleXtreme MALDI-TOF mass spectrometer. These components are arranged to produce and capture acoustic signals and conduct mass spectrometry assessments.

The speaker 320 transmits tailored acoustic signals through the liquid container 440, while the microphone array 330 records the propagated signals. These captured signals are relayed to the Raspberry Pi 3B 350 for data acquisition and initial processing. The UltrafleXtreme MALDI-TOF mass spectrometer furnishes authentic mass spectra for training and assessment purposes. The integration of these elements establishes a reliable infrastructure for data gathering and analysis.

Computer programs like Python are employed to generate and manipulate acoustic signals. In a sample scenario, the signals are composed of 21 sine waves spanning from 18 to 20 kHz and stored as WAV files. The implementation and training of the cGAN and FCNN models are executed using PyTorch.

Mass spectra are obtained utilizing the UltrafleXtreme MALDI-TOF mass spectrometer and processed via the flexAnalysis software to identify crucial peaks and generate the final mass spectral data.

FIG. 5 shows the process of identifying the medium implemented by the configuration and device of FIGS. 3A and 3B and FIGS. 4A-4B.

As shown, in step S501, acoustic signal is generated and emitted from the signal source, e.g., a speaker 120 or 320, passing through the liquid to be classified or identified, to the microphone array 130 or 330. In step S502, the microphones of the array receive signals and then transmit them to Raspberry Pi 3B 350 in which the system 110 is incorporated. The system 110 then implements the following steps:
Step S503: receiving, at the signal collection and fusion module/unit 111, signals transmitted through the medium from an array of signal receivers utilizing spatial diversity techniques, and performing weighted fusion of the received one or more signals to generate a fused signal;
Step S504: conducting, at the signal processing module/unit 113, signal processing including noise reduction and de-reverberation on the fused signal or received signal(s);
Step S505: extracting, at the extraction module/unit 115, the frequency-dependent attenuation characteristics of the medium from the signals;
Step S506: correcting and compensating, at the container compensation module/unit 117, for the frequency-dependent attenuation characteristics of the medium in a container, using a container compensation model;
Step S507: obtaining, at the mapping module/unit 119, mass spectra from the frequency-dependent attenuation characteristics of the medium by utilizing a deep learning model, which could be the cGAN, or autoencoders or Transformer-based networks;
Step S508: identifying, at the identification module/unit 121, the medium using a trained convolutional neural network based on the mass spectra.

Based on the above-described method and system or device, the preferred embodiments of the present application has demonstrated great performances.

Table 1 details the performance outcomes of the preferred embodiments within the present application concerning the detection of industrial alcohol adulteration among diverse beverage types. The system exhibits notable accuracy, precision, recall, and F1-score achievements across all scenarios. Notably, wine (W) attains a remarkable 99.25% accuracy rate under standard storage conditions, showcasing the system's adeptness in identifying tainted wine samples. Even under suboptimal storage conditions (Imp.), the system sustains its efficacy, with wine maintaining a stable accuracy of 98.25%. This resilience suggests the herein provided system's capability to withstand environmental variances like temperature fluctuations and humidity, which might influence the liquid's physical attributes. The system's high precision and recall values underscore its efficacy in minimizing false positives and false negatives, ensuring dependable detection of industrial alcohol adulteration.

TABLE 1
Industrial Alcohol Adulteration Detection Performance

| | Acc. | Prec. | Rec. | F1 |
|---|---|---|---|---|
| Type | (%) | (%) | (%) | (%) |
| W | 99.25 | 99.50 | 99.00 | 99.25 |
| B | 97.75 | 99.00 | 96.59 | 97.78 |
| S | 97 [symbol] 50 | 98 [symbol] 50 | 96 [symbol] 57 | 97 [symbol] 52 |
| Br | 96.25 | 97.00 | 95.57 | 96.28 |
| Wh | 96 75 | 97 50 | 96 06 | 96 77 |
| V | 98.50 | 99.00 | 98.02 | 98.51 |
| W (Imp.) | 98.25 | 98.00 | 98.49 | 98.25 |
| B (Imp.) | 97 25 | 97 00 | 97 49 | 97 24 |
| S (Imp.) | 96.75 | 98.00 | 95.61 | 96.79 |
| Br | 96.50 | 98.00 | 95.15 | 96.55 |
| Wh | 96 25 | 97 00 | 95 57 | 96 28 |
| V (Imp.) | 98.00 | 99.50 | 96.60 | 98.03 |

Note:
W = Wine, B = Beer, S = Chinese white spirit, Br = Brandy, Wh = Whisky, V = Vodka.
(Imp.) indicates improper storage conditions. [symbol]
indicates data missing or illegible when filed

Table 2 exhibits the performance metrics of the preferred embodiments within the present application in recognizing adulteration with lower-cost brands. The findings illustrate the system's consistent high accuracy and F1-scores across various beverage categories. For instance, wine (W) achieves a noteworthy F1-score of 99.50% under standard conditions, highlighting the system's proficiency in identifying subtle alterations in liquid composition due to adulteration with cheaper brands. Even under adverse storage conditions, the F1-score for wine marginally reduces to 99.25%, still indicating commendable performance. This underscores the system's reliability in detecting adulteration practices involving the blending of inferior products, crucial for upholding product integrity and consumer trust.

TABLE 2
Cheaper Brand Adulteration Detection Performance

| | Acc. | Prec. | Rec. | F1 |
|---|---|---|---|---|
| Type | (%) | (%) | (%) | (%) |
| W | 99.50 | 99.50 | 99.50 | 99.50 |
| B | 97.25 | 97.00 | 97.49 | 97.24 |
| S | 98 [symbol] 25 | 99 [symbol] 50 | 97 [symbol] 07 | 98 [symbol] 27 |
| Br | 98.00 | 98.00 | 98.00 | 98.00 |
| Wh | 99.00 | 99.00 | 99.00 | 99.00 |
| V | 98.50 | 99.00 | 98.02 | 98.51 |
| W (Imp.) | 99.25 | 99.50 | 99.00 | 99.25 |
| B (Imp.) | 97 00 | 97 00 | 97 00 | 97 00 |
| S (Imp.) | 98 75 | 98 00 | 99 49 | 98 74 |
| Br | 97 75 | 99 50 | 96 14 | 97 79 |
| Wh | 98 75 | 98 00 | 99 49 | 98 74 |
| V (Imp.) | 98.00 | 99.50 | 96.60 | 98.03 |

Note:
W = Wine, B = Beer, S = Chinese white spirit, Br = Brandy, Wh = Whisky, V = Vodka.
(Imp.) indicates improper storage conditions. [symbol]
indicates data missing or illegible when filed

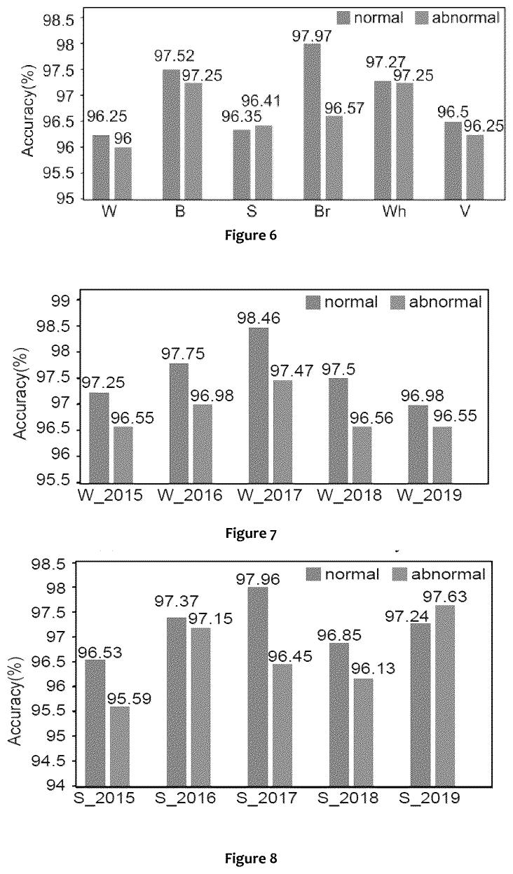

FIG. 6 shows the accuracy of brand recognition under normal and abnormal storage conditions for diverse beverage varieties. The preferred embodiments within the present application demonstrated high accuracy levels across all beverage types, showcasing its efficacy in distinguishing brands based on their distinct molecular compositions. Notably, wine (W) achieves a 99.25% accuracy rate under typical conditions and 98.25% under unfavourable conditions. This precision implies the system's reliable brand identification capability, critical for validating product authenticity and combating counterfeits. The minor decline in accuracy under abnormal storage conditions suggests the system's resilience and effectiveness despite environmental influences.

FIGS. 7 and 8 illustrate the accuracy of production year authentication for wine and Chinese white spirit across different vintages and storage conditions. The system consistently maintains high accuracy levels, exceeding 96% for all evaluated years. Specifically, for wine, as shown in FIG. 7, the peak accuracy of 98.46% is reached for the 2017 vintage, showcasing the system's aptitude in discerning nuances variations in beverage chemical compositions arising from aging and production fluctuations across years. Accurate year verification holds particular significance for products like wine and spirits, where vintage can significantly impact quality and market worth. The system's robust performance under varied storage conditions underscores its reliability for practical deployment.

In summary, the present application provides a method and system that enhanced liquid authentication by correlating acoustic signals with mass spectra, thereby offering molecular-level insights for precise identification. A cross-shaped microphone array is employed to effectively address positional variability, complemented by an adaptive container compensation algorithm to counteract the influence of diverse container characteristics. To facilitate dependable molecular-level classification, the system mapped acoustic signals to mass spectra through the integration of cGANs. Extensive experimental assessments confirmed that the present application achieved an average F1-score of 97.89%, with accuracy levels ranging from 95.35% to 98.25% across various container materials and storage conditions, ensuring robust and trustworthy liquid authentication.

It should be understood that the modules/units and steps presented above are for illustrative and preferred embodiment purposes. It is possible to eliminate certain steps outlined above while still achieving the objective of identifying the target medium. However, it should be understood that deviating from the preferred embodiment may result in a performance level that is not as optimal as demonstrated in the preferred scenario. For example, in an alternative embodiment, the system 100 of FIG. 1 is configured to remove the container compensation module/unit and thus the method of FIG. 5 is configured to remove step s506 accordingly. In the alternative embodiment, the objective of identifying the medium can still be reached, although the performance may be degraded.

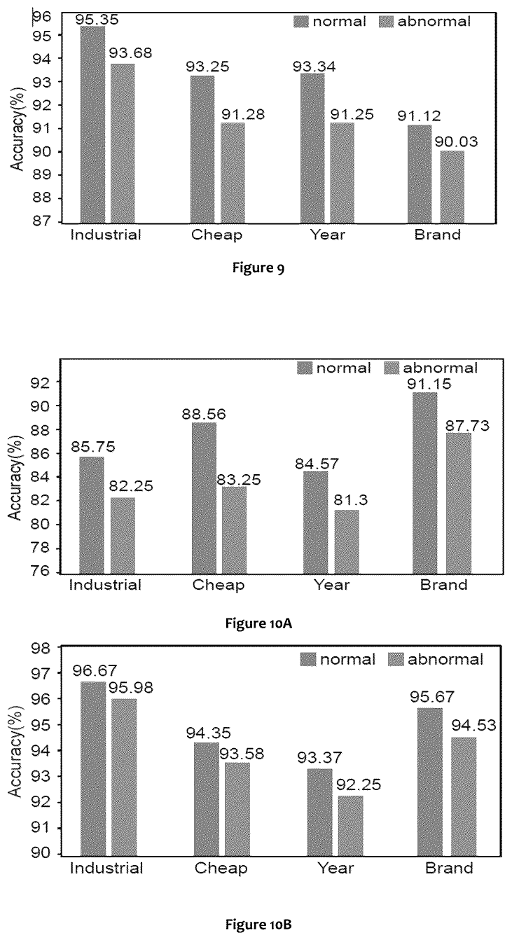

As shown in FIG. 9, there is a notable decrease in accuracy when the compensation module/unit is excluded, particularly under adverse storage conditions. In the context of industrial alcohol adulteration detection, the accuracy notably decreases from 95.35% to 93.68% in the absence of the compensation model. This decline emphasizes the significance of considering the impact of the container on the transmission of acoustic signals. Containers possess material characteristics and shapes that can introduce distortions capable of masking or modifying the acoustic profiles of the liquids. The container compensation model serves to effectively alleviate these influences, thereby augmenting the dependability and precision of the system.

In addition to the depiction in FIG. 9, which illustrated the difference between the system with and without the compensation module, FIGS. 10A and 10B further show the performance contrast between setups with and without the microphone array. FIG. 10A shows accuracy with single microphone and speaker under normal and improper storage conditions. FIG. 10B shows accuracy with five-microphone array under normal and improper storage conditions.

According to another alternative embodiment of the present application, only one microphone/receiver, instead of an array, is utilised to capture the signal. The signal is then processed according to steps S504-S508 as shown in FIG. 5 by a system selectively comprising the extraction module/unit 115, the mapping module/unit 119 and the identification module/unit 121. Alternatively, the system may further selectively include one or more of the signal processing module/units 113 and the container compensation module/unit 117. However, the performance may be degraded in certain circumstance compared with the system including the receiver array.

The accuracy attained in both configurations under normal and adverse storage conditions is demonstrated in FIGS. 10A and 10B. With the utilization of the five-microphone array, the system achieves heightened accuracy across all tasks, underscoring the efficacy of the array design in mitigating positional fluctuations and multipath effects. Notably, the accuracy of industrial alcohol adulteration detection reaches 96.67% with the array, surpassing the 85.75% achieved with a single microphone under standard conditions. This notable enhancement highlights that capturing signals from multiple spatially distributed microphones bolstered the system's resilience and dependability by averaging out noise and interference, while offering enhanced coverage of the acoustic field surrounding the container.

According to another alternative embodiment of the present application, cGAN is not integrated in the mapping module/unit when mapping the AATC to the corresponding mass spectra. It is still possible to identify the target medium such as liquid; however, the performance may be degraded.

Table 3 delineates the system's performance with and without cGAN integration for year classification, brand identification, and adulteration detection, evaluated under typical and adverse storage conditions. The tabulated results consistently favour the system's performance with the integration of cGAN, particularly in intricate classification tasks such as year verification and under unfavourable storage conditions. For instance, wine year verification accuracy surges from 89.75% to 96.82% with cGAN under adverse conditions, while adulteration detection improved from 91.50% to 97.71% under similar conditions, showing cGAN's performance in managing environmental fluctuations and subtle variations in liquid composition.

TABLE 3
Impact of GAN on Detection Accuracy

| | With | Only |
|---|---|---|
| | GAN | AATC |
| Task | (%) | (%) |
| Year (W, Normal) | 97.59 | 92.75 |
| Year (W, Imp.) | 96.82 | 89.75 |
| Brand (Normal) | 96.98 | 93.60 |
| Brand (Imp.) | 96.62 | 88.20 |
| Adulteration | 98.04 | 94.30 |
| Adulteration (Imp.) | 97.71 | 91.50 |

The technical solution of this application provides a broad spectrum of applications. It can be effectively utilized for liquid authentication across various industries including food and beverage safety, drug authentication, and quality control in chemical manufacturing processes. Moreover, it is well-suited for detecting counterfeit products, particularly in the realm of high-value consumer goods like spirits and perfumes.

Furthermore, the present application holds potential for widespread implementation in diverse fields such as environmental monitoring (e.g., water quality analysis) and biosafety (e.g., detection of hazardous chemicals or pathogens). Additionally, its applicability extends to healthcare, facilitating non-invasive detection of body fluids, thereby advancing diagnostic capabilities in the medical domain.

1. A method for identifying a medium, comprising:
receiving one or more wave-based signals emitted from one or more signal sources and transmitted through the medium;
extracting wave propagation characteristics of the medium from the signals;
transforming the extracted characteristics into a multi-dimensional spectral representation through computational analysis;
identifying the medium with a trained analysis system at least based on the generated spectral representation.

2. The method of claim 1, wherein the step of receiving one or more wave-based signals further comprising capturing the signals with an array of spatially distributed signal receivers, selecting the captured signals for weighted fusion and then performing the weighted fusion of selected captured signals to obtain at least one fused signal.

3. The method of claim 2, wherein the weight assigned to each selected signal for signal fusion is determined with a normalized signal-to-noise ratio approach.

4. The method of claim 3, wherein the weight assigned to each selected signal is computed as the ratio of its signal-to-noise ratio to the total signal-to-noise ratio of all selected captured signals.

5. The method of claim 2, wherein the array of spatially distributed signal receivers is configured as a cross-shaped array, circular array, or spherical array.

6. The method of claim 1, further comprising conducting signal processing including noise reduction and/or de-reverberation on the signals before extracting the wave propagation characteristics of the medium from the signals.

7. The method of claim 6, wherein the noise reduction is conducted through a denoising algorithm employing a discrete wavelet transform, and the de-reverberation is conducted through a weighted prediction error algorithm.

8. The method of any of preceding claims, wherein the one or more wave-based signals are acoustic signals, and the extracting step further includes performing a fast Fourier transform on the fused signal, extracting a frequency domain amplitude at each frequency, and obtaining an acoustic absorption and transmission curve as the wave propagation characteristics of the medium by normalising the extracted frequency domain amplitude by amplitudes of originally emitted wave-based signals.

9. The method of any of preceding claims, further comprising using a container compensation model to correct and compensate for the wave propagation characteristics of the medium accommodated in a container.

10. The method of claim 9, wherein the container compensation model utilizes a container transfer function defined as the ratio of a wave propagation characteristics of the medium with container influence to a wave propagation characteristic of the medium without container influence to perform the compensation.

11. The method of claim 10, wherein the container transfer function is learned through training a neural network regression model designed to transfer container characteristics, and wherein the neural network regression model includes an input layer receiving container characteristics, hidden layers incorporating nonlinear activation functions, and an output layer generating estimated wave propagation characteristics, with mean squared error as the loss function for training.

12. The method of any of preceding claims, wherein the computational analysis is conducted through a deep learning model including conditional generative adversarial network, or autoencoders or Transformer-based networks.

13. The method of claim 12, wherein the conditional generative adversarial network comprises a generator and discriminator, wherein the generator is configured to use the wave propagation characteristics as input to produce mass spectra as the spectral representation, and the discriminator is configured to classifies mass spectra based on the wave propagation characteristics of the medium.

14. The method of any of preceding claims, wherein the trained analysis system includes a fully connected neural network comprising an architecture with an input layer, three hidden layers, and an output layer, and wherein the input layer receives a spectrum vector representing the intensity of a specific mass-to-charge ratio, the three hidden layers consist of 512, 256, and 128 neurons, each using the ReLU activation function to introduce nonlinearity, and the output layer uses a Softmax activation function to output a probability distribution over the identification.

15. A device for identifying a medium, comprising a processor configured to execute any one of the methods as defined in claims 1-14.

16. The device of claim 15, further comprising:
an array of signal receivers for capturing signals transmitted through the medium.
